(19) United States
(12) Patent Application Publication
(10) Pub. No.: US 2026/0216535 A1
(43) Pub. Date: Jul. 30, 2026

(54) TRANSPORT SYSTEM FOR A VERY HIGH ENERGY ELECTRON (VHEE) BEAM

(71) Applicant: Yeda Research And Development Co. Ltd., Rehovot (IL)

(72) Inventors: Victor Armand MALKA, Tel Aviv (IL); Atul SENGAR, Rehovot (IL)

(21) Appl. No.: 19/147,024

(22) PCT Filed: Feb. 25, 2024
(86) PCT No.: PCT/IL2024/050211

§ 371 Date: Jul. 10, 2025

Related U.S. Application Data
(60) Provisional application No. 63/448,712, filed on Feb. 28, 2023.

(51) Int. Cl. A61N 5/10; G21K 1/02; G21K 1/08; G21K 5/04

(52) U.S. Cl. CPC A61N 5/1078; A61N 5/1045; G21K 1/025; G21K 1/08; G21K 5/04; A61N 2005/1089

(57) ABSTRACT

A transport line for a very high energy electron (VHEE) beam with a wide energy spread produced by a laser plasma accelerator (LPA) includes three quadrupoles and a collimator. The three quadrupoles focus the VHEE beam vertically and horizontally to provide a very high energy dose at a pre-defined depth within a body. The collimator is located after the three quadrupoles and filters out low energy particles.

### CROSS REFERENCE TO RELATED APPLICATIONS

This application claims priority from U.S. provisional patent application 63/448,712, filed on Feb. 28, 2023, which is incorporated herein by reference.

### FIELD OF THE INVENTION

The present invention relates to medical applications generally and to radiotherapy in particular.

### BACKGROUND OF THE INVENTION

Radiotherapy involves providing a particle or a radiation beam of some kind directed to a cancer tumor in the body. The various particle and radiation beams used today include ion beams and X-ray beams. Radiotherapy is designed to deliver a desired dose into the cancer tumor, while minimizing the dose in healthy tissues or in organs at risk.

FIG. 1, to which reference is now made, shows an exemplary radiotherapy machine 100 which generates a beam 104 to irradiate a tumor 102, deep inside a body 106. In order to avoid significant damage to healthy cells along a path 108, beam 104 should only be focused at the tumor depth D. However, beam 104 may lose energy as it moves through body 106 to tumor 102 and therefore, machine 100 must adjust the dose accordingly.

Unfortunately, machines to produce ion or proton beams require a lot of space and are quite expensive. Hadron therapy is, therefore, used most of the time, and particularly for complex cases. However, since their dose deposition is sensitive to inhomogeneities in body 106, there is a risk that the hadron beam will irradiate healthy tissue.

X ray beams, on the other hand, deliver a peak dose close to the skin surface. To irradiate deep tumors requires multiple-angle irradiation.

### SUMMARY OF THE PRESENT INVENTION

There is therefore provided, in accordance with a preferred embodiment of the present invention, a transport line for a very high energy electron (VHEE) beam with a wide energy spread produced by a laser plasma accelerator (LPA) includes three quadrupoles and a collimator. The three quadrupoles focus the VHEE beam vertically and horizontally to provide a very high energy dose at a pre-defined depth within a body. The collimator is located after the three quadrupoles and filters out low energy particles.

Moreover, in accordance with a preferred embodiment of the present invention, the collimator is a trapezoidal collimator or a trapezoidal collimator connected to a cylindrical collimator.

Further, in accordance with a preferred embodiment of the present invention, the three quadrupoles are equidistantly located one after each other.

Still further, in accordance with a preferred embodiment of the present invention, a first distance from a last quadrupole of the three quadrupoles and the body is equidistant to a second distance from the last quadrupole and a middle quadrupole of the three quadrupoles.

Moreover, in accordance with a preferred embodiment of the present invention, the pre-defined depth is within the vicinity of a tumor in the body.

Further, in accordance with a preferred embodiment of the present invention, the VHEE beam is a 250 MeV beam.

Finally, in accordance with a preferred embodiment of the present invention, an entrance dose at a skin of the body is less than 50% of a peak dose at the pre-defined depth.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of a prior art radiotherapy machine;

FIGS. 2A and 2B are a simplified isometric and side illustrations of a laser plasma accelerator machine (LPAM), constructed and operative in accordance with a preferred embodiment of the present invention;

FIG. 3A is a schematic illustration of a quadrupole, useful in the system of FIG. 2A;

FIG. 3B is a schematic illustration of a trapezoidal collimator, useful in the system of FIG. 2A;

FIG. 4 is a graphical illustration of the energy spectrum in number of particles per MeV within the transport line of FIG. 2A;

FIGS. 5 and 6 are graphical illustrations of the effect of the collimator of FIG. 3B, where FIG. 5 graphs on-axis dose profiles when a body is close by and FIG. 6 graphs on-axis dose profiles in a transport line when the body is further away; and

FIG. 7 is a schematic illustration of a combined collimator, formed of the trapezoidal collimator of FIG. 3B followed by a cylindrical collimator.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicant has realized that laser plasma accelerators (LPAs), which accelerate electrons, are less expensive and require less space than hadronic machines. However, they are not ideal. The higher the electron beam energy, the deeper the beam will penetrate the body. However, the energy scatters as it moves through the body.

Applicant has realized that, by focusing the beam inside the body, the entry dose can be reduced. Focusing improves longitudinal dose deposition by moving the higher peak dose to the tumor and lowering the entrance dose, thereby sparing healthy cells on the path to the tumor from damage.

However, a beam transport line (e.g. a magnetic focusing system controlling the electron beam) can only be optimized for a specific energy, and, in LPAs, the electron beam energy spectrum is broad, its energy distribution is large and its beam is divergent. When this broad spectrum beam passes through a magnetic focusing system, lower energy particles reach the body without being focused deeper into the patient and, as a result, the beam delivers an unwanted dose at the entrance to the body where there are healthy cells.

Applicant has further realized that a transport line for a very high energy electron (VHEE) beam, produced by an LPA, may include quadrupoles for focusing in both the vertical and horizontal planes and a collimator to stop unwanted ionizing side radiations that otherwise would affect the cells at the path entrance.

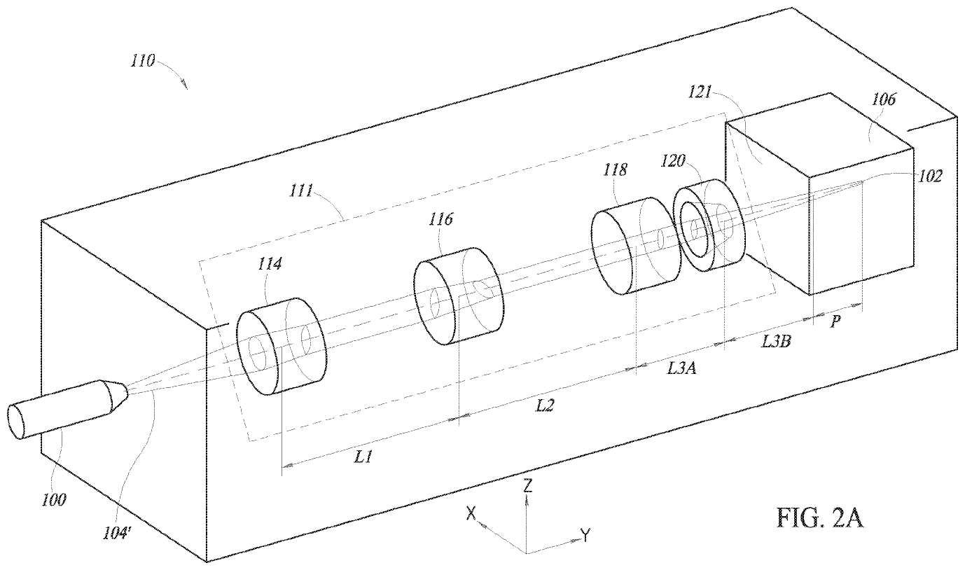

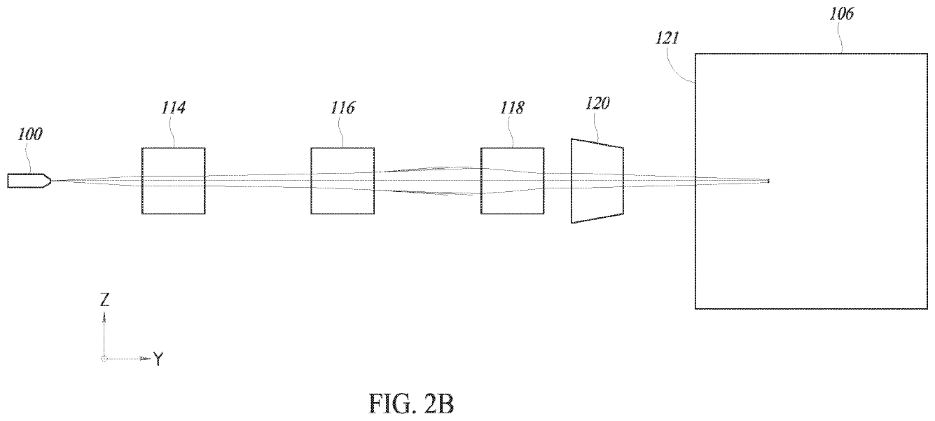

Reference is now made to FIGS. 2A and 2B, which illustrates an exemplary laser plasma accelerator machine (LPAM) 110, constructed and operative in accordance with a preferred embodiment of the present invention. FIG. 2A shows a simplified isometric view of LPAM 110 while FIG. 2B shows a simplified side view of LPAM 110.

LPAM 110 comprises an LPA 100, to generate a very high energy electron (VHEE) beam 104′, for example, of 250 MeV, with a wide energy spread, and a beam transport line 111 to focus electron beam 104′ within body 106. It will be appreciated that other types of VHEE beams are possible and are included in the present invention.

In accordance with a preferred embodiment of the present invention, beam transport line 111 may comprise 3 quadrupoles (114, 116, 118) to focus electron beam 104′ in a vacuum and a collimator 120. In one embodiment, outer quadrupoles 114 and 118 may focus electron beam 104′ in a vertical plane Y-Z while middle quadrupole 116 may focus electron beam 104′ in a horizontal plane X-Y. FIG. 2B indicates the focusing of quadrupole 114 in the vertical Z-Y plane, the scattering in that plane after quadrupole 116 and its refocusing by quadrupole 118.

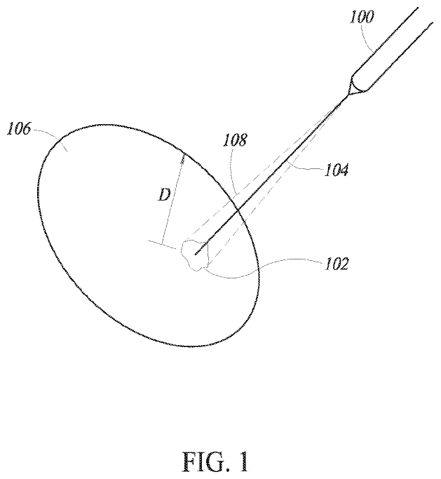

As mentioned hereinabove, a beam with a wide energy spread will deposit the energy of the lowest energy particles shortly after entering the body. This is not desired, as it may not only harm healthy cells but it may also reduce peak dose depth. Collimator 120 may be placed after quadrupole 118 to filter out the lower energy particles, thus significantly reducing the dose of electrons at the skin of body 106. The remainder of beam 104′ may be focused at a desired penetration depth p, such as the expected depth of a tumor, such as tumor 102 of FIG. 1. Thus, beam 104′ may be focused in the vicinity of the tumor 102.

In the embodiment of FIGS. 2A and 2B, quadrupoles 114, 116 and 118 may be equally spaced apart, with a distance L1 between quadrupoles 114 and 116, a distance L2 between quadrupoles 116 and 118, and a distance L3 between quadrupole 118 and body 106. For example, L1=L2=L3=30 cm. The penetration depth p for this embodiment is about 10 cm.

Collimator 120 may be placed within distance L3, such that there is a distance L3A between quadrupole 118 and collimator 120 and a distance L3B between collimator 120 and body 106. In one embodiment, distances L3A and L3B are the same. For example, they may be equal to 15 cm.

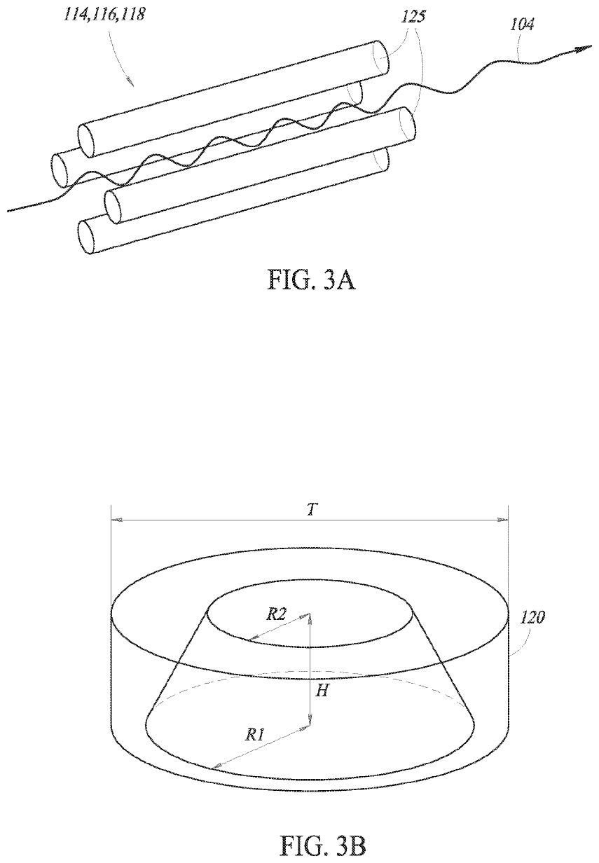

As shown in FIG. 3A to which reference is now briefly made, quadrupoles 114, 116 and 118 may be any suitable quadrupole and may be formed of four parallel magnetic tubes 125 carrying current thereon. Two opposing tubes 125 carry direct current while the other two tubes 125 carry an alternating current. Together, the currents focus beam 104 in the appropriate plane.

As can be seen in FIG. 3B, to which reference is now made, collimator 120 may be formed as a trapezoidal bore hole within a cylinder having a thickness T. Trapezoidal bore hole may have a frustum or trapezoidal shape with a larger radius R1 facing quadrupole 118 and an exit radius R2 facing body 106. Such a shape may reduce the entrance dose from beam 104′ to one which is quite close to a mono-energetic beam dose profile. This shape may restore the focusing effect of beam 104′, keeping the very high energy particles focused at the penetration depth and keeping the lower energy particles from scattering at the entrance to body 106.

In the embodiment for a 250 MeV beam, collimator 120 may have the following parameters:

$${R}_{1} = 2 cm ;$$

$${R}_{2} = 1 cm ;$$

$$H = 5 cm ;$$

$$T = 3 cm .$$

Collimator 120 may be formed of any suitable material capable of stopping electrons, such as tungsten or an alloy of tungsten, such as tungsten-copper. Since tungsten is a heavy material, it generally may stop more electrons for its size.

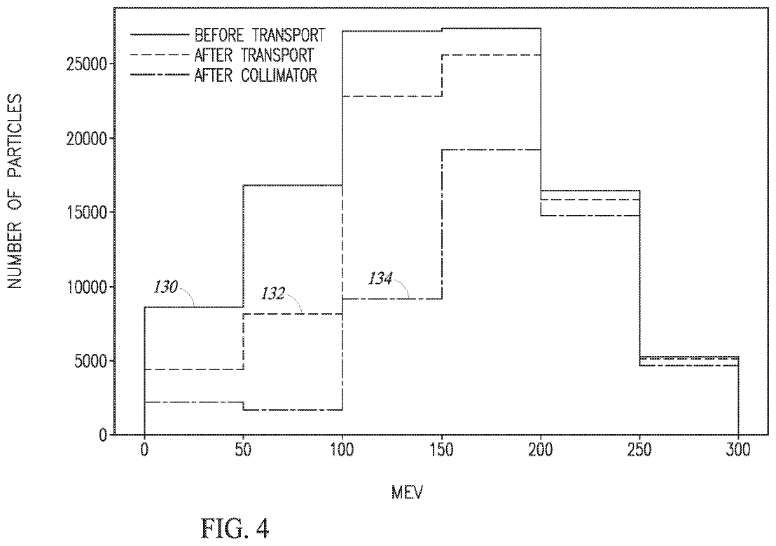

Reference is now made to FIG. 4, which graphs the energy spectrum in number of particles per MeV within transport line 111. Graph 130 plots the spectrum of the injected beam (output from LPA 100) while graph 132 plots the spectrum of the output of quadrupole 118. Note that there are less energetic particles in graph 132, as the transport line is optimized for a 250 MeV electron beam. Graph 134 plots the spectrum after collimator 120. Note that, in graph 134, particles with energies below 150 MeV are reduced while particles below 100 MeV are almost removed from the spectrum.

It will be appreciated that the behavior of stopping lower energetic particles does not depend on the energy spectrum injected into the transport line from LPA 100. In addition, the dimensions of collimator 120 may be changed depending on realistic beam parameters. It will be appreciated that inventive transport line 111 may provide significant flexibility to stop unwanted ionizing radiation, at relatively low cost.

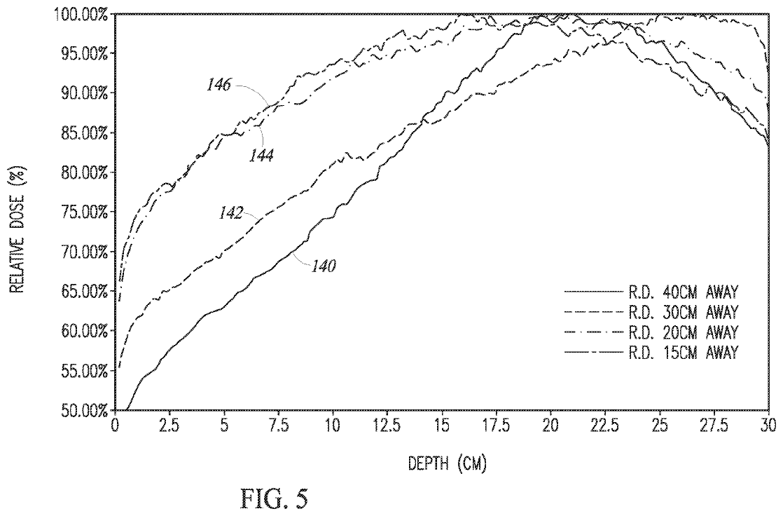

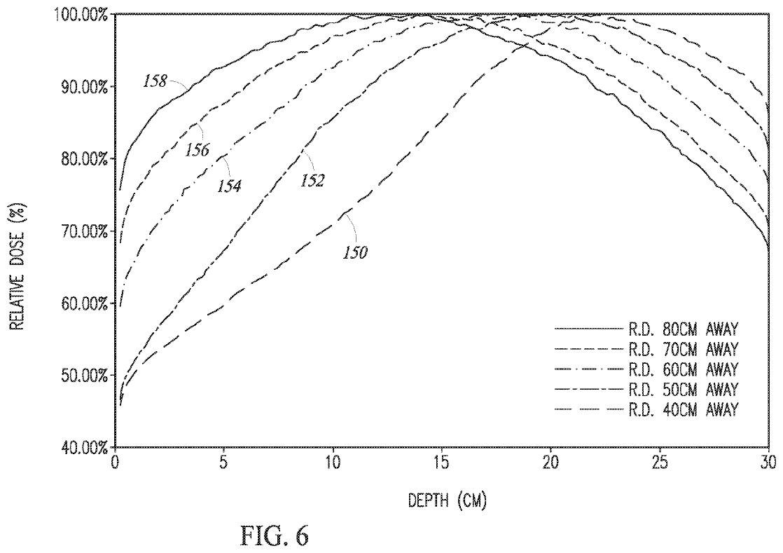

Reference is now made to FIGS. 5 and 6, which show the effect of collimator 120, where FIG. 5 graphs on-axis dose profiles in transport line 111 for a short L3 (i.e. when body 106 is relatively close to last quadrupole 118) while FIG. 6 graphs on-axis dose profiles in transport line 111 when L3 is larger.

FIG. 5 graphs dose profiles for body 106 located at different distances (15-40 cm) from last quadrupole 118 (i.e. for different lengths of L3). Graph 140 shows the relative dose at 40 cm, graph 142 shows the relative dose at 30 cm, graph 144 shows the relative dose at 20 cm and graph 146 shows the relative dose at 15 cm. It can be seen that the peak dose depth increases as body 106 is moved away from quadrupole 118. Thus, graph 140 has a peak dose depth of about 20 cm while graph 146 has a peak dose depth of about 15.5 cm.

However, it should be noticed that, as body 106 is closer to last quadrupole 118, the entrance dose increases (graph 140 at 40 cm has less than a 50% dose at the entrance (0 cm depth) while graph 146 at 15 cm has a 67% dose at the entrance).

FIG. 6 graphs dose profiles for body 106 located at different distances (40-80 cm) from last quadrupole 118 but with collimator 120 located halfway between last quadrupole 118 and body 106. Graph 150 shows the relative dose at 40 cm, graph 152 shows the relative dose at 50 cm, graph 154 shows the relative dose at 60 cm, graph 156 shows the relative dose at 70 cm and graph 158 shows the relative dose at 80 cm. In this case, peak dose depth increases as body 106 is moved towards from quadrupole 118. Thus, graph 150 has a peak dose depth of about 22 cm while graph 158 has a peak dose depth of about 12 cm.

However, in the embodiment of FIG. 6, with collimator 120, the entrance dose increases at greater distances (graph 150 at 40 cm has less than a 50% dose at the entrance while graph 158, much further away at 80 cm, has a 76% dose at the entrance). These results are more desirable.

FIG. 6 indicates that, with collimator 120 removing unwanted ionizing radiation, the wider energy spread VHEE produced by LPA 100 may be used for radiotherapy. Moreover, the on-axis dose is sufficient for radio-therapy applications.

It will be appreciated that laser plasma accelerator machine 110 may provide an inexpensive radiotherapy system that utilizes less space. LPAM 110 may provide a penetration depth which is deeper with a higher amount of energy to the electron beam. By focusing the beam inside the body with the help of collimator 120, electron scattering can be reduced. Focusing improves penetration depth and localizes higher peak doses into a smaller volume with a lower entrance dose. The result is reduced damage to healthy cells in the vicinity of the tumor.

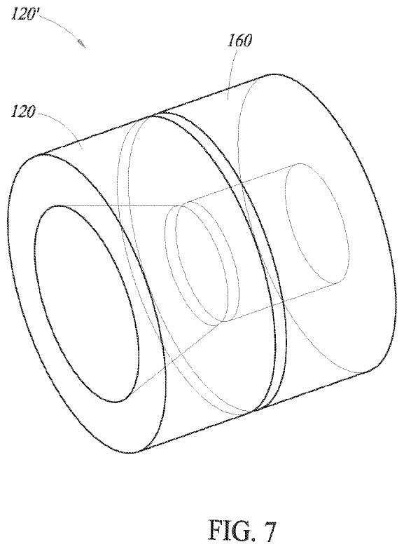

An alternative embodiment shown in FIG. 7 to which reference is now made, may utilize a combined collimator 120′, formed of trapezoidal collimator 120 followed by a cylindrical collimator 160, with a bore hole of 1 cm length. Cylindrical collimator 160 may counter small angle scattering that can happen at the edges of trapezoidal collimator 120 collimator 120. Cylindrical collimator 160 may have the same thickness T as collimator 120 and may be connected to it.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A transport line for a very high energy electron (VHEE) beam with a wide energy spread produced by a laser plasma accelerator (LPA), the system comprises:
three quadrupoles to focus said VHEE beam vertically and horizontally to provide a very high energy dose at a pre-defined depth within a body; and
a trapezoidal collimator after said three quadrupoles to filter out low energy particles.

2. (canceled)

3. The transport line of claim 1 and also comprising a cylindrical collimator connected after said trapezoidal collimator.

4. The transport line of claim 1 wherein said three quadrupoles are equidistantly located one after each other.

5. The transport line of claim 1 wherein a first distance from a last quadrupole of said three quadrupoles and said body is equidistant to a second distance from said last quadrupole and a middle quadrupole of said three quadrupoles.

6. The transport line of claim 1 wherein said pre-defined depth is within a vicinity of a tumor in said body.

7. The transport line of claim 1 wherein said VHEE beam is a 250 MeV beam.

8. The transport line of claim 1 wherein an entrance dose at a skin of said body is less than 50% of a peak dose at said pre-defined depth.

9. The transport line of claim 1 wherein said trapezoidal collimator is formed of tungsten or a tungsten-copper alloy.

10. The transport line of claim 4 wherein said trapezoidal collimator is located halfway between said last quadropole and said body.

11. The transport line of claim 1 wherein said pre-defined depth is within the vicinity of a tumor in said body.

12. The transport line of claim 6 and wherein said trapezoidal collimator has an entrance radius of 2 cm and an exit radius of 1 cm.
